(19) United States
(12) Patent Application Publication
(10) Pub. No.: US 2004/0147459 A1
(43) Pub. Date: Jul. 29, 2004

(54) Method and composition for maintaining urinary tract health in the face of infections

(76) Inventors: Joseph Oneal, Irving, TX (US); Gary White, Irving, TX (US)

Correspondence Address: CARSTENS YEE & CAHOON, LLP, P O BOX 802334, DALLAS, TX, 75380

(21) Appl. No.: 10/691,423

(22) Filed: Oct. 22, 2003

Related U.S. Application Data
(60) Provisional application No. 60/420,696, filed on Oct. 23, 2002.

(51) Int. Cl. A61K 31/70; A61K 35/78

(52) U.S. Cl. 514/23; 424/771

(57) ABSTRACT

The sugar mannose has been used to maintain urinary tract health in the face of E. coli infections. An optimal dose is disclosed to be of one teaspoon (two grams) three times a day for one to two weeks or until symptoms subside. The maintenance dosage for prophalaxis is one-half teaspoon (1 gram) 1 to two times per day. Children's dosages are cut in half. For women who experience UTIs after sexual relations, one teaspoon is taken an hour prior to intimate relations and an additional one teaspoon immediately afterwards. It is further disclosed to use any of an extract of Crataeva nurvala, white willow bark, and pollen extract in conjunction with the mannose to provide further effect.

### RELATED APPLICATIONS

This application claims the benefit, under 35 U.S.C. § 119(e)(1), of U.S. Provisional Application Serial No. 60/420,696, filed Oct. 23, 2002, which is incorporated herein by this reference.

### BACKGROUND OF THE INVENTION

1. Technical Field

The present invention relates to a non-pharmacological method of maintaining urinary health when faced with urinary tract infections.

2. Description of the Related Art

The female urinary tract, illustrated in FIG. 1, consists of the two kidneys 102, which remove water and impurities from the bloodstream to form urine, the two ureters 104, which carry the urine from the kidneys to the bladder 106, and the urethra 108, a short tube which provides the outlet from the bladder 106 to the outside world. The male urinary tract is similar, except that the urethra is much longer and exits through the penis.

Urinary tract infections (UTIs) are a common problem in many persons. Bacteria that are a normal part of the lower intestines will sometimes be inadvertently transferred from the anal region to the urethra. Although most of these bacteria will be flushed out by urination, some bacteria are able to attach to the lining of the urinary tract and remain. Women are more prone to UTIs, due to the short length of the female urethra and the nearness of the urethral opening to the anal and vaginal areas. Sexual activity can also cause a transfer of bacteria and is a cause of chronic infections in a number of women. Although men get fewer UTIs during most of their life, they become more prone to UTIs as they age, especially if prostate enlargement is present. An infection that remains in the urethra is called “urethritis”. If the infection travels to the bladder, it becomes “cystitis” while, if it manages to reach the kidneys, it is known as “nephritis” or “pyelonephritis”. Symptoms include inflammation and swelling of the tissue, which results in the urge to urinate, with pain and burning on urination. In conventional medical practice, the normal treatment of urinary tract infections (UTIs) is a prescription of antibiotics. Typically an antibiotic is chosen that will pass into the urine in a potent form. The symptoms of a UTI may be gone in only a few days, but treatment often lasts up to two weeks, in order to prevent a recurrence from resistant bacteria. In chronic cases, the patient may be placed on low doses of antibiotics for an extended period of time. An undesirable side effect of antibiotic treatment is that normal bacteria in the body are destroyed along with the bacteria causing the infection. This can allow other problems to develop, such as the vaginal yeast infections many women experience when taking an antibiotic. Additionally, if the antibiotic does not completely destroy the infection, or if the patient discontinues the treatment too early, the infection can recur, often in a more virulent form. The development of resistant strains of bacteria is a major issue in health care today, so finding ways to avoid making the problem worse has great appeal, both for patients and for those in the medical field.

A folk remedy for UTIs that has evoked interest both inside and outside the medical profession calls for drinking cranberry juice to fight or prevent infections. Patients experiencing difficulties with UTIs are encouraged to drink large amounts of cranberry juice until the infection is eliminated. This remedy is not always effective, but it has provided relief in many cases. It was originally believed that the acid in cranberry juice helped destroy the bacteria, although the true mechanism is different, as will be explained.

It is medically accepted that in about 90% of the cases of urinary tract infections, the bacteria involved are Escherichia coli (E. coli), one of the bacteria normally found in the colon. When this bacteria is introduced into the urethra, E. coli is able to attach to the epithelial lining, allowing it to ascend through the urinary tract. The E. coli bacteria have hair-like projections called fimbriae on their cell walls. These fimbriae contain a protein that causes them to bind to certain sugars. Unfortunately, the epithelial cells in the urinary tract manufacture the sugar mannose internally and their surface contains this molecule. Binding of the fimriae to these sugar molecules provides the mechanism by which the E. coli can attach and avoid being swept out of the body during urination.

In studies of the mechanism by which cranberry juice helped resolve urinary infections, it was discovered that the sugar fructose, contained in cranberry juice, can also bind to E. coli. Whenever the bacteria bind to the fructose, they can be eliminated. Since then, researchers have found that the sugar, D-mannose, the same sugar produced in the urinary tract lining, works ten times more effectively than fructose in binding to E. coli and inhibiting its attachment. Mannose, which has a chemical formula of C₆H₁₂O₆, is stereoisomeric with glucose. This means that on a molecular level, glucose and mannose are mirror images of each other. As is often true of stereoisomeric molecules, only one of the forms, glucose in this case, is useable by the body as a food. The mannose tastes sweet when ingested, but it will not be broken down in the body for fuel. Instead, it is passed into the urine in an intact form and is then excreted. An E. coli bacteria, when surrounded by molecules of mannose in the urine, will more often bind to the mannose in the urine than to the mannose in the epithelial cells, allowing it to be eliminated naturally. Those few bacteria that remain can then be better handled by the body's natural defenses, the white blood cells.

However, maintaining urinary health with mannose is still a relatively new development, with much to still be learned. It would be desirable to establish the optimal amounts of mannose and the optimal timing, as well as determining other ingredients that may further aid the action of mannose.

### SUMMARY OF THE INVENTION

The present invention discloses the use of D-mannose in the following dosages for optimal results: one teaspoon (two grams) three times a day for one to two weeks or until symptoms subside. Mannose is safe for diabetics, pregnant women, and the elderly, as it appears to be virtually impossible to overdose with mannose.

The present invention further discloses the combination of D-mannose with one or more of the following adjuncts: willow bark, an extract of Crataeva nurvala, and pollen extract, each of which provides further benefit to the urinary tract.

### BRIEF DESCRIPTION OF THE DRAWINGS

A more complete understanding of the method and composition of the present invention may be had by reference to the following detailed description when taken in conjunction with the accompanying drawings, wherein:

FIG. 1 diagrammatically shows the urinary tract.

FIG. 2 is a table showing dosages for different ages and conditions.

FIG. 3 is a flowchart regarding the use of mannose when faced with a urinary tract infection.

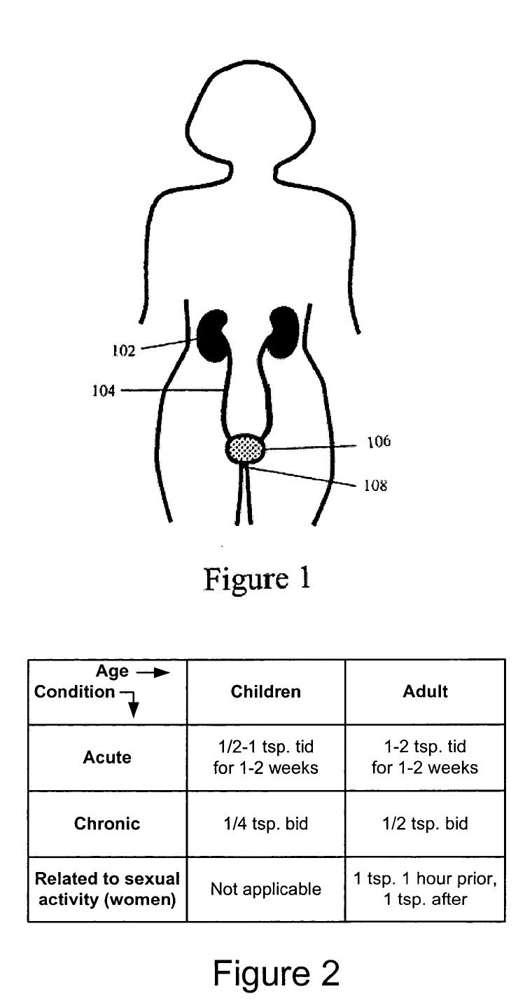

### DETAILED DESCRIPTION OF THE INVENTION

In the prior art, it has been suggested to take 1 gram (½ teaspoon) of mannose every two to three hours while awake and at night whenever awake. Because it is difficult for many people to remember a medication that must be taken this often, it is herein disclosed to change this model, as disclosed in the table in FIG. 2. For acute infections, the method is to give a 2-gram (1 teaspoon) dose of mannose three times a day with meals. The maintenance dosage is one-half teaspoon (1 gram) one to two times per day. Children's dosages are half of the adult dosage. For women who experience UTIs after sexual relations, one teaspoon is used an hour prior to intimate relations and an additional one teaspoon immediately following. This regimen provides a quantity of mannose sufficient to remove a majority of E. coli in the urinary tract, while improving the ease of use and compliance. These dosages have been shown to be effective in doctor-run trials.

It is further disclosed that the addition of any items from the group that includes an extract of Crataeva nurvala, white willow bark, and pollen extract further aids in the dealing with urinary tract problems. Each of these additives will be discussed separately. In the presently preferred embodiment of the invention, the mannose is supplied in a capsule. Each capsule contains 500 mg of D-mannose, 10 mg of Cratavin™, a standardized extract of Crataeva nurvala, 15 mg of while willow bark standardized extract containing 2.25 mg of salicin, and 5 mg of Cernilton 63™, a standardized pollen extract, in an inert base of magnesium stearate. The suggested use is 4 capsules three times a day, which provides the recommended 2 grams of D-mannose. The dosages can also be taken in a powder form.

Cratavin™ is available from SAMI Labs, Inc of Piscataway, N.J. It is an extract of the root bark and stem bark of the evergreen tree Crataeva nurvala, with the major chemical constituent being lupeol. The tree C. nurvala is indigenous to India, where it is also known by its Sanskrit name of Varuna. Traditional Indian medicine (Ayurveda) has long considered this extract to be beneficial in dealing with urinary disorders. Although much of the attention in using Cratavin with urinary disorders has centered on urolithiasis (urinary tract stones), Cratavin has also been studied in the management of urinary tract infections. When given to a number of chronic urinary infection cases, 17% of patients who received a 4-week course of C. nurvala were symptom free and their urine was free of microorganisms and pus. It is believed that this is due to the fact that C. nurvala is anti-inflammatory (i.e., reduces the inflammation due to the infection) and has a tonic effect (i.e., it produces healthy muscle tone) in the bladder to help prevent occurrences of urine retention. Both of these properties provide further help in clearing up infections of the urinary tract.

Willow bark consists of the bark of 2-3-year-old branches, harvested during early spring, of Salix alba L., S. purpurea L., S. fragilis L. and other comparable Salix species. This herb is commonly used for fever and pain. The therapeutic benefit is attributed to salicylates, which are similar to aspirin (acetylsalicylic acid), although they are metabolized differently in the body. The standardized extract used contains 9 mg of salicin in a 60 mg dosage and can make the sufferer more comfortable, as well as fight the inflammation.

Another substance that is beneficial in urinary tract infections is flower pollen. Cemitin™ is a standardized flower pollen extract, supplied by Graminex of Saginaw, Mich. The pollen is from a variety of plants and contains both water-soluble and fat-soluble fractions in a ratio of 20:1.

Pollen helps to bioregulate organism functions such as the immune system, lipid metabolism, and blood cholesterol level. It also helps regulate the function of the prostate and both enhances peak pressure during urination and decreases retention after urination. The precise mode of action is not known, although experimental studies suggest that it has anti-inflammatory and anti-androgenic¹ properties, again properties that aid in the management of urinary tract infections. ¹ Decreases the activity of male sex hormones, which are known to participate in prostate hypertrophy

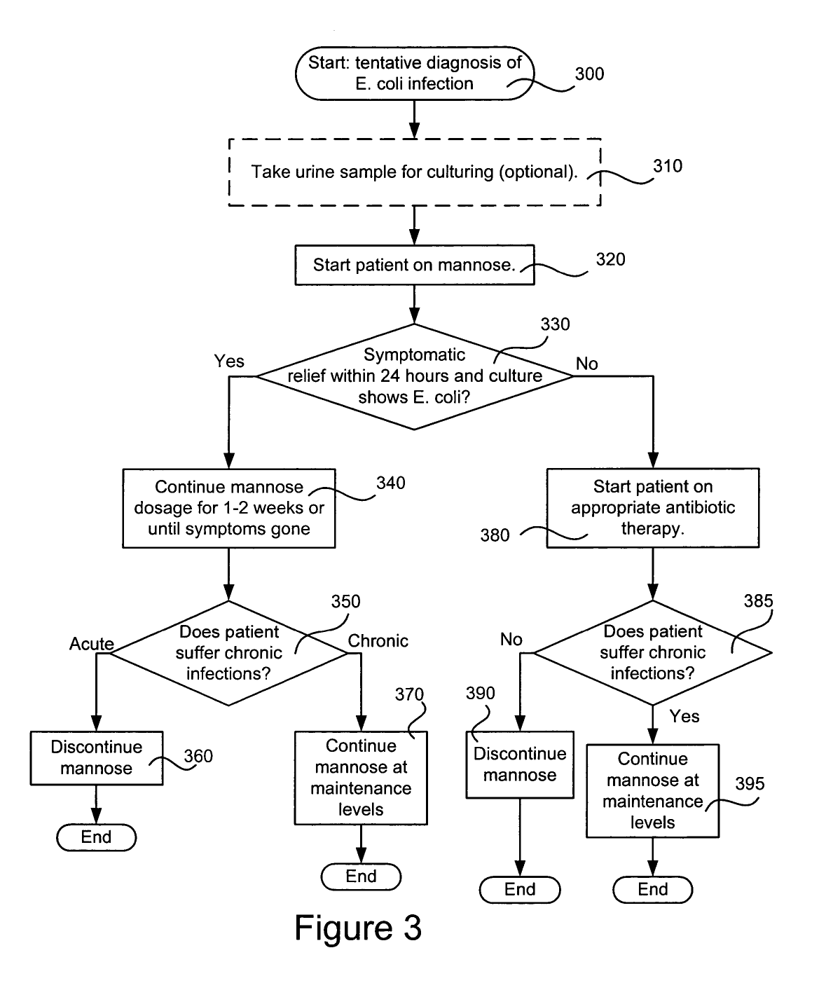

FIG. 3 shows a flowchart for the management of a urinary tract infection. Since 80 percent of UTIs are caused by E. coli, the doctor can being with a tentative diagnosis of E. coli infection (step 300). Many doctors will want to take a urine specimen for culturing (step 310), so that if another organism is the culprit, treatment with an appropriate antibiotic can be started as soon as possible. In cases of chronic infections that have proven to be E. coli, the clinician may optionally proceed without culturing. The patient is started on an appropriate dosage of mannose, according to their age and the severity of the condition (step 320). For an E. coli infection, there should be marked improvement within 24 hours. If so (step 330), the patient may be instructed to continue the dosage for 1-2 weeks (step 340) to be sure the E. coli are eliminated. However, unlike antibiotics, a patient who stops taking mannose too early is not encouraging the development of a more resistant strain. Since the mechanism is purely mechanical, the use of mannose can simply be restarted. If the infection is acute, i.e., a one-time infection, (step 350), the patient is instructed to discontinue the mannose (step 360) once symptoms are completely gone. If however, the patient suffers from chronic UTIs (step 350), instructions generally recommend continuing on a maintenance dose after the current episode is resolved (step 370).

When the infections turns out to be something other than E. coli, the patient will have no significant relief within 24 hours and a culture, if done, will be positive for another organism (step 330). In this case, the doctor can start the patient on an appropriate antibiotic (step 380) to combat the organism found. At the same time, the doctor will want to know if the patient has recurring problems with UTIs (step 385). If so, the patient can be placed on a maintenance dosage of mannose (step 395); if not, the mannose can be discontinued (step 390).

In summary, it is possible to adjust the dosages and administration times of mannose to encourage better compliance. The use of other ingredients having beneficial effects on the health of the urinary tract can provide additional help in fighting infections.

We claim:

1. A method of maintaining urinary tract health in the face of an infection, comprising the steps of:
administering a dosage of one to two teaspoons of D-mannose to the patient three times a day with meals for one to two weeks or until the symptoms subside.

2. The method of claim 1, wherein said D-mannose is administered orally.

3. The method of claim 1, wherein said D-mannose is administered as a powder.

4. The method of claim 1, wherein said D-mannose is administered in a capsule.

5. The method of claim 1, wherein said D-mannose is administered in a capsule containing herbs that affect the urinary tract.

6. The method of claim 1 further comprising administering at least one of a group consisting of an extract of Crataeva nurvala, willow bark, and pollen extract simultaneously with said D-mannose.

7. The method of claim 1, further comprising administering each of an extract of Crataeva nurvala, willow bark, and pollen extract simultaneously with said D-mannose.

8. A method of dealing with a urinary tract infection in a patient comprising the steps of:
administering a therapeutically effective dosage of D-mannose and a therapeutically effective dosage of at least one of the group of an extract of Crataeva nurvala, willow bark, and pollen extract simultaneously with said D-mannose to a patient.

9. The method of claim 8, wherein said dosages are administered in a capsule.

10. The method of claim 8, wherein each dose contains approximately 2 grams of mannose.

11. The method of claim 8, wherein a dose is administered three times a day with meals.

12. The method of claim 8, wherein each dose contains approximately 40 mg of Cratavin™ or its equivalent.

13. The method of claim 8, wherein each dose contains approximately 60 mg of willow bark standardized extract or its equivalent.

14. The method of claim 8, wherein each dose contains approximately 20 mg of pollen extract or its equivalent.

15. A composition for maintaining urinary tract health in the face of infections, comprising:
a therapeutically effective dosage of D-mannose; and
a therapeutically effective dosage of at least one of the group of an extract of Crataeva nurvala, willow bark, and pollen extract.

16. The composition of claim 15, wherein said composition comprises a therapeutically effective dosage of each of an extract of Crataeva nurvala, white willow bark, and pollen extract.

17. The composition of claim 15, wherein said composition is provided in capsules, each of said capsules comprising 500 mg of D-mannose, 10 mg of an extract of Crataeva nurvala, 15 mg of white willow bark containing 2.25 mg of salicin, and 5 mg of pollen extract or their equivalents.
